(19) United States
(12) Patent Application Publication
(10) Pub. No.: US 2013/0077738 A1
(43) Pub. Date: Mar. 28, 2013

(54) COLLIMATOR, DETECTOR ARRANGEMENT, AND CT SYSTEM

(71) Applicant: Siemens Aktiengesellschaft, Munich (DE)

(72) Inventors: Björn KREISLER, Erlangen (DE); Bodo REITZ, Forchheim (DE)

(73) Assignee: Siemens Aktiengesellschaft, Munich (DE)

(21) Appl. No.: 13/626,141

(22) Filed: Sep. 25, 2012

(30) Foreign Application Priority Data
Sep. 26, 2011 (DE) 102011083394.3

(51) Int. Cl. G21K 1/02; A61B 6/03

(52) U.S. Cl. 378/7; 378/149

(57) ABSTRACT

A collimator for a detector, such as an x-ray detector of a CT system, includes a plurality of collimator modules. At least one of the plurality of collimator modules includes at least two outer collimator walls and at least one inner collimator wall (1a). The at least one inner collimator wall (1a) has a plurality of steps.

### CROSS-REFERENCE TO RELATED APPLICATION(S)

The present application hereby claims priority under 35 U.S.C. §119 to German patent application number DE 10 2011 083 394.3 filed Sep. 26, 2011, the entire contents of which are hereby incorporated herein by reference.

### BACKGROUND

1. Field

Example embodiments relate to collimators for detectors, particularly for x-ray detectors of computed tomography (CT) systems, that have a multiplicity of collimator modules, having at least two outer collimator walls and at least one inner collimator wall. Example embodiments further relate to detector arrangements having collimators of such kind and to CT systems having detector arrangements of such kind.

2. Description of Related Art

The relevant information during image reconstruction in CT systems is found in the attenuating of x-rays coming from the x-ray tube's focus. The detector elements of a detector of the CT system that are sensitive to the x-radiation are—without further technical measures—sensitive to x-rays impinging within a large angle range. X-ray sources outside the x-ray tube therefore also contribute to a detector element's signal. In CT systems, scattered radiation principally constitutes additional x-ray sources of such kind outside the x-ray tube. Said scattered radiation gives rise to an additional signal contribution during image reconstruction. However, said additional signal contribution results in a poorer signal-to-noise ratio so that disruptive image artifacts may arise if the proportion of scattered radiation changes locally, meaning for respectively adjacent detector elements.

The aim in using what is termed an anti-scatter collimator (ASC) is to limit the detector elements' angular acceptance to the tube-focus direction and reduce the scattered radiation's contribution so that the reconstructed images will, in the end, have improved quality. ASCs known to date and employed in CT systems are of one-dimensional design and limit the angular acceptance only in the phi direction. Two-dimensional ASCs (2D ASCs), which limit the angular acceptance in both the phi and the z direction, are still in the development stage.

Conventional 2D ASCs have a minimum wall thickness of 85 μm, which for production reasons cannot be further reduced. Said 2D ASCs are of modular design. In width they typically cover one module. An option is for a plurality of 2D ASCs (typically two to four) to be arranged side by side in the z direction, but it is alternatively also possible to produce 2D ASCs, each covering one module. The 2D ASCs have a continuous collimator wall on all four external sides. The edge pixels of two adjacent detector elements or, as the case may be, the collimator walls located on a module's edge will thereby effectively have twice the collimators' wall thickness. The scattered radiation in the detector elements' edge regions will, for that reason, be suppressed to a greater extent than in the case of detector elements located centrally on a module. That edge effect will give rise to annular image artifacts during image reconstructing. That problem does not arise in the case of 1D ASCs as compared with 2D ASCs. Through the ASC's being constructed from single collimator walls, the modules could be designed such that both for peripheral detector elements and for centrally located detector elements there is one collimator wall for each side. No structural solution to that problem has yet been found for 2D ASCs.

In the operation of a CT system having an x-ray tube it has hitherto been possible to significantly reduce the aforementioned artifacts only by what is termed balancing using a symmetrical water phantom. It has not yet been demonstrated whether that would also meet the objective in clinical applications. The effect is stronger in a CT system's dual-source operating mode and cannot be resolved by balancing alone.

### SUMMARY

Example embodiments provide collimators in the case of which edge effects in the detector elements' edge regions will be avoided so that image reconstructing that is as free as possible from artifacts will be possible in CT systems.

The inventors have recognized that the edge effects and hence the artifacts arising during image reconstructing in a CT system can be drastically reduced by embodiments of the collimator walls of the collimator. To achieve that, the central collimator walls of a two-dimensional collimator, for example a 2D ASC, are structured like the steps of a staircase. The individual steps are therein embodied as being smaller or, as the case may be, narrower from bottom to top. A height of the steps is expediently embodied as being constant or substantially constant. That step shape can be realized by, for example, conventional collimator production methods. To simplify the production methods for collimators, one step corresponds to one layer, for example, with the collimator walls being assembled from about six to about twenty individually produced layers. The collimator walls' step or staircase shape can extend preferably in both the phi and the z direction.

A module's central or, as the case may be, inner collimator walls may be embodied as having a plurality of steps of different thickness. The individual steps' width therein increases from top to bottom so that the inner collimator walls are shaped like a staircase. The steps can therein extend in the phi and the z direction. In keeping with a staircase shape, the bottommost step is formed as the widest and the topmost step as the narrowest. Each step is formed from one layer, for example, to simplify the collimator walls' production. A step can, though, alternatively also be formed from a plurality of layers or a layer can have a plurality of steps. The narrow, topmost step can be produced having a width or, as the case may be, wall thickness that is the minimum possible during production. The minimum width is for production reasons approximately 80 μm. The bottommost and widest step is referred to also as the foot; it serves to stabilize the collimator wall on the detector elements and defines the aperture of the individual image elements.

The outer collimator walls may have two steps. The two steps can be formed from a thick foot as the bottom step and a single high step. Said high step can be produced having the minimum possible width, for example, approximately 80 μm. The high step can furthermore be structured from a plurality of layers.

What is understood within the scope of this patent application by the localizing terms “outer” and “inner” as applied to a collimator module is an edge region bordering adjacent collimator modules and a central region, respectively.

Because of the staircase-shaped collimator walls inside the collimator modules and the two-step collimator walls on the outer edge of the collimator modules it is possible to harmonize the average wall thickness, which is to say the width, of the collimator walls on all sides of all detector elements without, in doing so, sacrificing one of the collimator's characteristics that is critical and also advantageous for image reconstructing. The collimator can be produced using various fabrication technologies, for example, by employing a molding method or by building the collimator up from thin layers, because the minimum wall thickness of about 80 μm will not be undershot.

Collimators according to example embodiments have a continuous collimator wall on all external sides, which ensures the component's and module's simple manageability as well as their stability. Compared with conventional collimator walls, the width of the foot remains constant or substantially constant because the additional material of the collimator walls, which is to say the wider layers or steps, are located in the region of the projection of the foot's width in the phi direction. The grid ratio, which is to say the ratio of the height of the collimator walls to the maximum width of the collimator walls on the foot, referred to the detector elements, is consequently unchanged and constant or substantially constant. Nor, furthermore, will the necessary precision in producing and positioning the collimator walls then be affected. The last two points, namely the constant grid ratio and the positioning of the collimator walls, would not apply if the width of the inner collimator walls is to be evenly increased. Attenuating of the scattered radiation will, though, additionally be significantly increased overall owing to the effectively wider collimator walls.

The inventors accordingly propose improving a collimator for a detector, particularly for an x-ray detector of a CT system, that has a multiplicity of collimator modules, having at least two outer collimator walls and at least one inner collimator wall. The at least one inner collimator wall has a plurality of steps. A collimator of such kind will enable the scattered radiation of the x-rays in a CT system to be effectively filtered so that artifacts due to scattered radiation will be suppressed and/or almost totally prevented during image reconstruction.

The inventors accordingly also propose improving a collimator for a detector, particularly for an x-ray detector of a CT system, that has a plurality of collimator modules. At least one of the plurality of collimator modules has at least two outer collimator walls and at least one inner collimator wall. The at least one inner collimator wall has a plurality of steps.

What is to be understood by an outer collimator wall is a collimator wall located at an edge of the collimator module, whereas the inner collimator walls are accordingly located between the outer collimator walls inside the collimator module. At least one inner collimator wall is advantageously provided and at least two (e.g., three, four, or more) inner collimator walls. The inner collimator walls inventively have a plurality of steps (e.g., three, four, or five) so that the shape realized is that of a staircase. The individual steps' width advantageously decreases from bottom to top. A bottommost step accordingly has a maximum width. For example the bottommost step is embodied as a foot. A topmost step furthermore has a minimum width. The outer collimator walls are embodied preferably in keeping with the conventionally known collimator walls, thus, for instance, having one wide foot as the bottommost step and a further, single, narrow step whose width is minimal. The inner and outer collimator walls may, alternatively, be embodied as being the same or substantially the same height, meaning that the sum of the individual steps of the inner and outer collimator walls is the same or substantially the same.

The steps of the collimator may be formed in both the phi and the z direction. The collimator walls' shape resembling that of steps or a staircase will then have been produced through a structure comprising layers arranged one upon the other and upwardly reducing in size.

The steps of the inner collimator walls may be the same or substantially the same height. It will consequently be advantageously easy to produce the collimator walls, meaning to form the steps. The steps may be between about 100 μm and about 500 μm, inclusive, high and more preferably between about 200 μm and about 400 μm, inclusive, high. The width of the individual steps, excepting the foot, may evenly decrease upwardly, for example such that the bottommost step is two or three times as wide as the topmost layer.

A topmost step of the collimator may have a minimum width in the range between about 50 μm and about 110 μm inclusive, between about 60 μm and about 100 μm, inclusive, or between about 70 μm and 90 μm, inclusive. In a more specific example, a width of the topmost step is approximately 80 μm, which corresponds to a minimum possible wall thickness in the case of conventional production techniques.

In another embodiment of the collimator, a bottommost step may have a maximum width in the range between about 150 μm and about 300 μm, inclusive, or between about 180 μm and about 220 μm, inclusive. The bottommost and widest step serves as what is termed a foot for stabilizing the collimator walls on the detector elements. The bottommost step is in an example embodiment two to three times as wide as the topmost, narrowest step.

The steps of different collimator walls are embodied as being of equal or substantially equal width and/or height. That will make it easier to produce the collimator walls. The collimator walls are produced preferably layer by layer. A typical collimator wall has between about five and about twenty layers that are, for example, molded individually. To further simplify the collimator walls' production, a step of a collimator wall corresponds to a layer of the collimator wall. In other embodiments, a step corresponds to a plurality of layers, for example two or three.

A layer can alternatively have a plurality of steps. The steps or layers are therein advantageously made of a single material. For example tungsten, molybdenum, tantalum, lead, copper, or metal alloys containing a high percentage of such metals are suitable as the material for the collimator walls. The walls can either be purely metallic or can include metal powder in a plastic matrix. The collimator material advantageously has a high atomic number.

Example embodiments also provide a detector arrangement that has at least one detector for absorbing radiation, in particular for absorbing x-radiation, and at least one inventive collimator having at least one of the above-described characteristics. The detector includes a multiplicity of detector elements. A plurality of detector elements will be covered on each of the collimator's collimator modules. The individual collimator walls are, for example, each located on the transitional regions of two adjacent detector elements.

Example embodiments also provide a computed tomography (CT) system having at least one of the above-described detector arrangements and by which tomographical recordings of an object being examined can be generated. Using the inventive collimators enables images to be reconstructed in the CT system advantageously virtually free from artifacts owing to the improved absorption of the scattered radiation by the step-shaped inner collimator walls.

### BRIEF DESCRIPTION OF THE DRAWINGS

Example embodiments will be more clearly understood from the description of the drawings.

In the figures, the following reference numerals/letters are employed: n, n+1: Collimator module; 1a: Inner collimator wall; 1b: Outer collimator wall; 2: Foot; 3: Step; 10: Detector element.

FIG. 1 shows a schematic representation of a cross-section through two collimator modules of a conventional, two-dimensional collimator on a plurality of detector elements;

FIG. 2 shows a schematic representation of a cross-section through two collimator modules of a two-dimensional collimator on a plurality of detector elements, according to an example embodiment;

FIG. 3 shows a schematic representation of a cross-section through a collimator wall according to an example embodiment; and

FIG. 4 shows a chart of a simulation of a scattered-radiation signal in relation to the primary signal.

### DETAILED DESCRIPTION

Various example embodiments will now be described more fully with reference to the accompanying drawings in which only some example embodiments are shown. Specific structural and functional details disclosed herein are merely representative for purposes of describing example embodiments. The present invention, however, may be embodied in many alternate forms and should not be construed as limited to only the example embodiments set forth herein.

Accordingly, while example embodiments of the invention are capable of various modifications and alternative forms, embodiments thereof are shown by way of example in the drawings and will herein be described in detail. It should be understood, however, that there is no intent to limit example embodiments of the present invention to the particular forms disclosed. On the contrary, example embodiments are to cover all modifications, equivalents, and alternatives falling within the scope of the invention. Like numbers refer to like elements throughout the description of the figures.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items.

It will be understood that when an element is referred to as being “connected,” or “coupled,” to another element, it can be directly connected or coupled to the other element or intervening elements may be present. In contrast, when an element is referred to as being “directly connected,” or “directly coupled,” to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Spatially relative terms, such as “beneath”, “below”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, term such as “below” can encompass both an orientation of above and below. The device may be otherwise oriented (rotated 90 degrees or at other orientations) and the spatially relative descriptors used herein are interpreted accordingly.

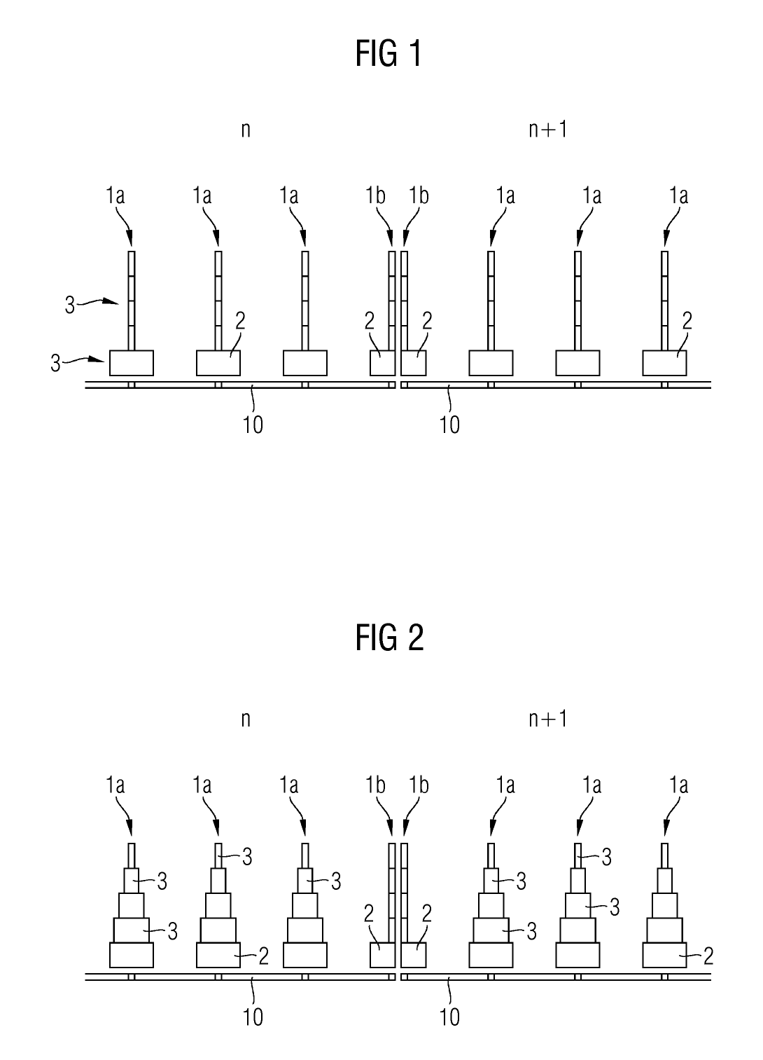

FIG. 1 is a schematic representation of a cross-section through two collimator modules n and n+1 of a two-dimensional collimator on a plurality of detector elements 10. Collimator modules n and n+1 are not shown in their entirety in that representation but only at their transitional region or, as the case may be, at the module boundaries with the other collimator module. Collimator modules n and n+1 each include a plurality of collimator walls 1a, 1b, meaning in each case one outer collimator wall 1b at the module boundaries, with only one module boundary and consequently only one outer collimator wall 1b being shown here, and three inner collimator walls 1a that are shown. Outer collimator walls 1b are each located in the edge region of collimator modules n and n+1, meaning at the module boundaries; inner collimator walls 1a are located inside collimator modules n and n+1, meaning in each case between outer collimator walls 1b. Detector elements 10 are located below collimator walls 1a, 1b. Collimator walls 1a, 1b are each positioned above the boundaries of two adjacent detector elements 10.

Collimator walls 1a and 1b each have a foot 2 for stabilized positioning on detector elements 10. Collimator walls 1a and 1b furthermore each have four equally wide layers on foot 2 embodied as the bottommost layer, with the four top layers being embodied as a step 3. Foot 2 is substantially wider than the layers or, as the case may be, second step 3, in this embodiment approximately seven times wider. Top step 3 has for production reasons a minimum width of approximately 80 μm.

Collimator walls 1a, 1b are shown in their conventional embodiment in the representation in FIG. 1. Inner and outer collimator walls 1a and 1b respectively are accordingly implemented as being equal or substantially equal, excepting foot 2 shortened towards adjacent outer collimator wall 1b. Because in each case two outer collimator walls 1b meet at the module boundary and at the same time the width of top step 3 cannot be further reduced, the width for detector elements 10 at the module boundaries is twice that of the other collimator walls 1a.

FIG. 2 is a schematic representation of a cross-section through two collimator modules n and n+1 of a two-dimensional collimator on a plurality of detector elements 10, according to an example embodiment. Detector elements 10 and the arrangement of outer and inner collimator walls 1b and 1a correspond to the embodiment shown in FIG. 1. Components that are the same are identified by the same reference numerals/letters. A more detailed description of components that have already been described has therefore been dispensed with.

Inner collimator walls 1a inventively have a step- or staircase-shaped structure with in this case five steps 3. Foot 2 forms bottommost step 3. The top four steps 3 on foot 2 are each formed from one layer. Each layer forms a step 3 in that embodiment. The width of steps 3 decreases evenly upwardly. Topmost step 3 has a minimal width of approximately 80 μm. Steps 3 have according to FIG. 2 been formed in the phi and the z direction. Steps 3 are according to FIG. 2 therein embodied as being rectangular so that each layer forms a cuboid in a layered arrangement.

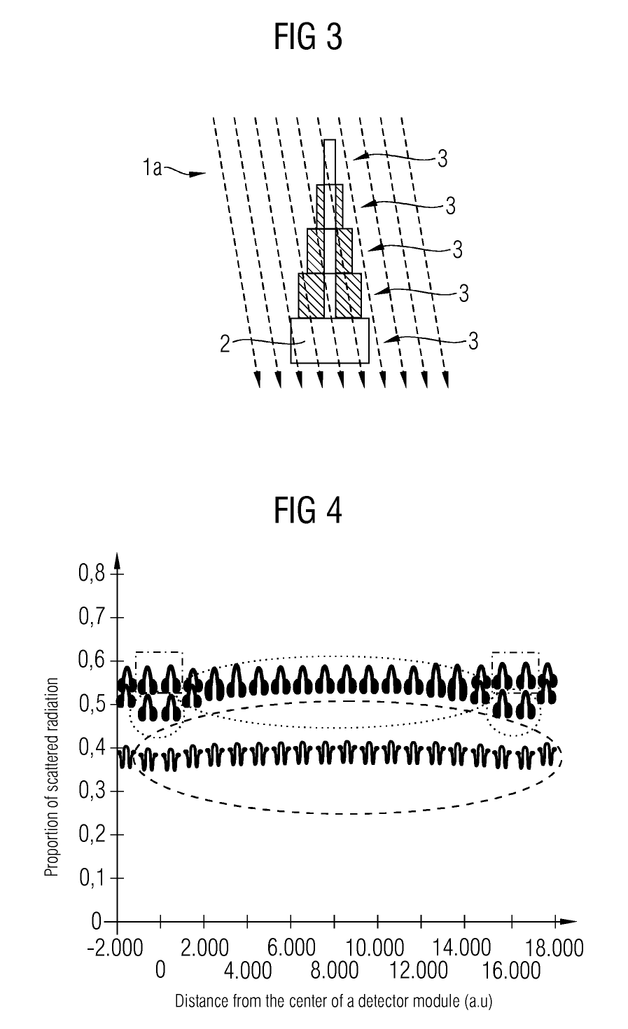

FIG. 3 is a schematic representation of a cross-section through a step-shaped inner collimator wall 1a, according to an example embodiment. A plurality of x-rays are additionally shown as dashed lines. The x-rays' course through collimator wall 1a and individual steps 3 can be seen therein. The additional material of inventive steps 3 is shown hatched and shaded for comparing the effective width of inner collimator wall 1a with a conventional (outer) collimator wall. The x-radiation impinges from above in the representation in FIG. 3, meaning from the direction of the narrowest, top step onto the collimator wall. For the x-radiation, the additional material is accordingly situated inside the line linking the leading edge and the foot. Hence only x-rays from the directions that would also be shielded in the case of a conventional collimator will be absorbed by the additional material. Twice as much material as in the case of the conventional collimator will on average be penetrated given a suitably selected width of steps 3. The collimator wall therefore has the same effect as the two stepless collimator walls 1b at the module boundaries, without increasing the dead zone.

FIG. 4 is a chart of the simulation of a scattered-radiation signal in relation to the primary signal in the detector center of a CT detector. Only an extract is shown. An image element's width is in this case 1,000 units. Inner collimator walls were in that example simulated at locations −2,000, −1,000, +1,000, +2,000, . . . 14,000, +15,000, +17,000, and +18,000, and various outer collimator walls at locations 0 and 16,000. The proportion of scattered radiation in the respective image element's signal is plotted as a function of the phi coordinate during scanning of a water phantom having a diameter of approximately 30 cm and a large Z coverage. The proportion of scattered radiation, meaning the ratio between direct radiation impinging on the detector element and the impinging scattered radiation, is plotted on the ordinate. A conventional two-dimensional collimator (see FIG. 1) covers the 0-to-16,000 range, with the collimator walls being situated at locations 0, 1,000, 2,000, . . . , 15,000, and 16,000. The module boundaries having two collimator walls are situated at 0 and 16,000.

The data points inside the dot-dashed rectangles are the simulation results from a two-dimensional collimator that is idealized, though not able to be produced, and in the case of which the two outer collimator walls in total have the same width as an inner collimator wall. Shown in the dotted circles are the data points of the simulation results from a conventional two-dimensional collimator (see FIG. 1) having conventional collimator walls. The difference between the detector modules' edge detector elements and the central detector elements is a few percentage points; it has image relevance and without corrections will result in annular artifacts. The simulation results for an inventive two-dimensional collimator (see FIG. 2) having step-shaped inner collimator walls are shown in a dashed frame. Overall, that is where the scattered radiation is suppressed best. Only minimal differences remain between central and edge detector elements so that the image artifacts will have been minimally to completely eliminated.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A collimator for a detector, the collimator comprising:
a plurality of collimator modules, at least one of the plurality of collimator modules including two outer collimator walls and at least one inner collimator wall, the at least one inner collimator wall having a plurality of steps.

2. The collimator as claimed in claim 1, wherein the plurality of steps are formed in the phi and z direction.

3. The collimator as claimed in claim 1, wherein the plurality of steps are the same height.

4. The collimator as claimed in claim 1, wherein a topmost of the plurality of steps has a minimum width in the range between about 50 μm and about 110 μm, inclusive.

5. The collimator as claimed in claim 1, wherein a bottommost of the plurality of steps has a maximum width in the range between about 150 μm and 300 μm, inclusive.

6. The collimator as claimed in claim 1, wherein a step is structured from at least one layer of a collimator material.

7. The collimator as claimed in claim 1, wherein the collimator walls include at least one of tungsten, molybdenum, tantalum, lead, copper, or metal alloys thereof.

8. The collimator as claimed in claim 7, wherein the collimator walls are purely metallic.

9. The collimator as claimed in claim 7, wherein the collimator walls include a metal powder in a plastic matrix.

10. A detector arrangement having at least one detector for absorbing radiation, the detector arrangement comprising:
at least one collimator as claimed in claim 1.

11. A CT system comprising:
at least one detector arrangement as claimed in claim 10.

12. The collimator as claimed in claim 1, wherein a topmost of the plurality of steps has a minimum width in the range between about 60 μm and about 100 μm, inclusive.

13. The collimator as claimed in claim 1, wherein a topmost of the plurality of steps has a minimum width in the range between about 70 μm and about 90 μm, inclusive.

14. The collimator as claimed in claim 1, wherein a bottommost of the plurality of steps has a maximum width in the range between about 180 μm and 220 μm, inclusive.

15. A collimator module for a collimator of a detector, the collimator module comprising:
two outer collimator walls; and
at least one inner collimator wall, the at least one inner collimator wall having a plurality of steps.

16. The collimator module as claimed in claim 15, wherein the plurality of steps are formed in the phi and z direction.

17. The collimator module as claimed in claim 15, wherein the plurality of steps are the same height.

18. The collimator module as claimed in claim 15, wherein a topmost of the plurality of steps has a minimum width in the range between about 50 μm and about 110 μm, inclusive.

19. The collimator module as claimed in claim 15, wherein a bottommost of the plurality of steps has a maximum width in the range between about 150 μm and 300 μm, inclusive.
